(19) United States
(12) Patent Application Publication
(10) Pub. No.: US 2026/0100249 A1
(43) Pub. Date: Apr. 9, 2026

(54) METHOD, ELECTRONIC DEVICE AND STORAGE MEDIUM

(71) Applicant: Bytedance Technology Ltd., Grand Cayman (KY)

(72) Inventors: Nima Shoghi Ghalehshahi, Los Angeles, CA (US); Yuxuan Liu, Los Angeles, CA (US); Yuning Shen, Los Angeles, CA (US); Robert Gregory Brekelmans, Los Angeles, CA (US); Quanquan Gu, Los Angeles, CA (US)

(21) Appl. No.: 19/417,665

(22) Filed: Dec. 12, 2025

(51) Int. Cl. G16B 30/00

(52) U.S. Cl. CPC G16B 30/00

(57) ABSTRACT

Embodiments of the present disclosure provides a method, an electronic device and a storage medium. In the method, a plurality of first residue features for a plurality of residues in the first conformation and a plurality of second residue features for a plurality of residues in a second conformation are determined based on a protein sequence and a first conformation, the protein sequence comprising a plurality of residues; the plurality of second residue features are updated based on temporal information and spatial information of the plurality of first residue features; and the second conformation is generated based on the updated plurality of second residue features.

### FIELD

Embodiments of the present disclosure mainly relate to the field of biology, and more particularly, to a method, an electronic device and a storage medium.

### BACKGROUND

Proteins are the core executors of life activities, and the realization of their functions relies not only on specific amino acid sequences but is also closely related to their dynamic conformational changes. This dynamic process is referred to as protein conformational dynamics. Conformational dynamics encompasses various motions, ranging from minor vibrations of local residues to large-scale conformational rearrangements of entire structural domains, and is key to understanding protein functional mechanisms, molecular recognition, and enzymatic catalytic efficiency.

The study of protein conformational dynamics has been widely integrated into various applied fields such as biomedicine and biotechnology. For example, in drug discovery, drug design strategies based on protein conformational dynamics are receiving increasing attention. By targeting the dynamic conformations of proteins rather than a single static structure, the specificity and efficacy of drugs may be significantly improved.

### SUMMARY

Embodiments of the present disclosure provide a method, an electronic device and a storage medium.

In a first aspect of the present disclosure, a method is provided. The method includes: determining, based on a protein sequence and a first conformation, a plurality of first residue features for a plurality of residues in the first conformation and a plurality of second residue features for a plurality of residues in a second conformation, the protein sequence comprising a plurality of residues; updating, based on temporal information and spatial information of the plurality of first residue features, the plurality of second residue features; and generating, based on the updated plurality of second residue features, the second conformation.

In a second aspect of the present disclosure, an electronic device is provided. The electronic device includes: at least one processor; and at least one memory, which is coupled to the at least one processor and stores instructions being executed by the at least one processor. The instruction, when executed by the at least one processor, causes the electronic device to: determine, based on a protein sequence and a first conformation, a plurality of first residue features for a plurality of residues in the first conformation and a plurality of second residue features for a plurality of residues in a second conformation, the protein sequence comprising a plurality of residues; update, based on temporal information and spatial information of the plurality of first residue features, the plurality of second residue features; and generate, based on the updated plurality of second residue features, the second conformation.

In a third aspect of the present disclosure, a non-transitory computer-readable storage medium is provided. The computer-readable storage medium has program instructions stored thereon. The program instructions, when executed by an apparatus, cause the apparatus to perform the method described in the first aspect of the present disclosure.

It is to be understood that the summary section is not intended to identify key or essential features of embodiments of the present disclosure, nor is it intended to be used to limit the scope of the present disclosure. Other features of the present disclosure will become easily comprehensible through the following description.

### BRIEF DESCRIPTION OF THE DRAWINGS

Some example embodiments will now be described with reference to the accompanying drawings, in which:

FIG. 1 illustrates a schematic diagram of an example environment in which embodiments of the present disclosure may be implemented;

FIG. 2 illustrates a flowchart of a method according to an embodiment of the present disclosure;

FIG. 3 shows a schematic diagram of a process for generating a conformation according to an embodiment of the present disclosure;

FIG. 4 shows a schematic diagram of a diffusion process according to an embodiment of the present disclosure;

FIG. 5 shows a schematic diagram illustrating the effect of joint attention according to an embodiment of the present disclosure; and

FIG. 6 illustrates a schematic block diagram of an example device that can be used to implement embodiments of the present disclosure.

Throughout the drawings, the same or similar reference numerals represent the same or similar elements, unless otherwise indicated.

### DETAILED DESCRIPTION OF EMBODIMENTS

Principle of the present disclosure will now be described with reference to some example embodiments. It is to be understood that these embodiments are described only for the purpose of illustration and help those skilled in the art to understand and implement the present disclosure, without suggesting any limitation as to the scope of the disclosure. The disclosure described herein can be implemented in various manners other than the ones described below.

In the following description and claims, unless defined otherwise, all technical and scientific terms used herein have the same meaning as commonly understood by one of ordinary skills in the art to which this disclosure belongs.

References in the present disclosure to “one embodiment,” “an embodiment,” “an example embodiment,” and the like indicate that the embodiment described may include a particular feature, structure, or characteristic, but it is not necessary that every embodiment includes the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

It shall be understood that although the terms “first” and “second” etc. may be used herein to describe various elements, these elements should not be limited by these terms. These terms are only used to distinguish one element from another. For example, a first element could be termed a second element, and similarly, a second element could be termed a first element, without departing from the scope of example embodiments. As used herein, the term “and/or” includes any and all combinations of one or more of the listed terms.

The terminology used herein is for the purpose of describing particular embodiments only and is not intended to be limiting of example embodiments. As used herein, the singular forms “a”, “an” and “the” are intended to include the plural forms as well, unless the context clearly indicates otherwise. It will be further understood that the terms “comprises”, “comprising”, “has”, “having”, “includes” and/or “including”, when used herein, specify the presence of stated features, elements, and/or components etc., but do not preclude the presence or addition of one or more other features, elements, components and/or combinations thereof. As used herein, “at least one of the following: <a list of two or more elements>” and “at least one of <a list of two or more elements>” and similar wording, where the list of two or more elements are joined by “and” or “or”, mean at least any one of the elements, or at least any two or more of the elements, or at least all the elements.

When predicting protein conformations, the prediction often needs to be started from a known conformation, and the motion trajectory of each atom of the protein at various time points is calculated according to the rules of motion. Due to the highly complex structure of proteins and the mutual influences between residues, this process involves substantial computational demands. In related art, based on the assumption that spatial and temporal dependencies can be completely separated, computational load is reduced by processing temporal influences independently from spatial influences. However, the true dynamics of proteins are full of non-separable spatiotemporal coupling. Consequently, conformations calculated in this way are inaccurate. Particularly when predicting a series of conformations over a longer time period, deviations accumulate increasingly, leading to highly inaccurate predicted conformations, thus making it impossible to predict a series of conformations over a long duration. Furthermore, when calculating spatial influences, a scenario can occur where residues in a preceding conformation can see residues in a subsequent conformation, which does not align with reality.

Embodiments of the present disclosure provide a method, an electronic device and a storage medium. The method, electronic device and storage medium involve updating the residues of the second conformation based on both the temporal information and spatial information of each residue in the first conformation. The second conformation generated in this manner is not only structurally reasonable itself, but the transition from the first conformation also more closely conforms to real physical dynamics laws. This directly enhances the accuracy of the second conformation. Moreover, since this method operates based on residue features, computational and memory overheads are reduced to some extent, enabling the prediction of conformations for larger proteins or over longer time scales.

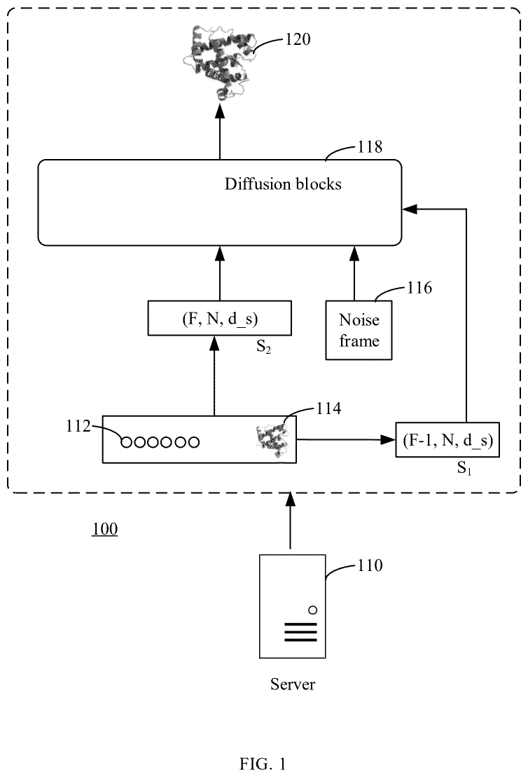

Embodiments of the present disclosure will be described in further detail below with reference to the accompanying drawings. FIG. 1 shows a schematic diagram of an example environment 100 in which embodiments of the present disclosure can be implemented. The example environment 100 includes a server 110. The server 110 may be deployed with a model (e.g., a transformer, a multimodal model capable of processing multimodal data, a diffusion model, and combinations thereof, etc.). This model may include multiple encoders, multiple diffusion blocks, and a decoder. In this embodiment, the method according to the embodiments of the present disclosure is executed by the server 110.

In some embodiments, the server 110 may acquire a protein sequence 112 comprising a plurality of residues and a first conformation 114. The protein sequence 112 may be a textual description of the plurality of residues, which may include information such as the name, attributes, and properties of each residue. The first conformation 114 serves as the starting point for predicting the second conformation 120 and includes the three-dimensional coordinates and rotational orientations of the plurality of residues in the first conformation 114.

In some embodiments, the server 110 may determine, based on the protein sequence 112 and the first conformation 114, a plurality of first residue features S₁ for the plurality of residues in the first conformation 114 and a plurality of second residue features S₂ for the plurality of residues in the second conformation 120. For example, the server 110 may use a first encoder to encode the protein sequence 112 and the first conformation 114 to obtain the plurality of second residue features S₂. In some embodiments, a preset time interval (e.g., 2 nanoseconds) is maintained between consecutive conformations. As shown in FIG. 1, the plurality of second residue features S₂ may be represented by 3 parameters (F, N, d_s), where F represents the sequence number of the frame or conformation corresponding to S₂, N represents a number of residues, and d_s represents the dimensionality of each residue feature. The time point of the frame or conformation may be inferred from its sequence number F and the preset time interval. During the conformation generation process, the number of residues N is consistent across all conformations and matches the number of residues in the protein sequence 112. In some embodiments, the dimensionality of each residue feature may be a vector with a length of 128. Similarly, the plurality of first residue features S₁ may be represented by 3 parameters (F-1, N, d_s), where F-1 indicates that the first conformation 114 is a previous conformation with respect to the second conformation 120.

The server 110 may use a second encoder to encode the first conformation 114 and protein sequence 112, thereby obtaining the plurality of first residue features S₁. The second encoder that is used to generate residue features without noise for a previous conformation, may have a different structure from the first encoder that is used to generate residue features with noise for a to-be-predicted conformation. These first residue features S₁ may extract spatial information (e.g., the position and rotational orientation of each residue) of the residues in the first conformation 114. In some embodiments, a first residue feature S₁ may indicate, at a starting time point, properties such as the characteristics, position, and rotational orientation of the fifth residue in the protein. For example, S₁ might indicate that counting from the N-terminus (the start of the polypeptide chain) to the C-terminus (the end), the fifth residue is Glutamine, located at position (1, 1, 1), with a rotational orientation being a specific direction in Rotation-space.

In some embodiments, the server 110 may update the plurality of second residue features S₂ based on the temporal information and spatial information of the plurality of first residue features S₁. Each diffusion block among the multiple diffusion blocks 118 may include one or more attention modules and a backbone module. The attention module is used to calculate attention weights between features, and the backbone module is used to generate the conformation. In this process, the temporal information may be incorporated in to S₁ by adjusting S₁ (e.g., rotating S₁ by one degree). The attention module calculates the attention weights of S₂ relative to S₁ and S₂ itself, enabling S₂ to be updated based on the temporal and spatial information contained within the features of each residue in the adjusted S₁.

In some embodiments, the server 110 may generate the second conformation 120 based on the updated plurality of second residue features. A noise frame 116 may be input into the backbone module within the multiple diffusion blocks 118. This backbone module uses the plurality of second residue features S₂ to denoise the noise frame 116. The dimensionality of a residue of the noise frame 116 may include two components: dimensionality in three-dimensional space (3D) and dimensionality in rotational space (4D). Through iterative denoising, an accurate second conformation 120 may be obtained. The updated plurality of second residue features S₂ records the features representing the accurate positions and rotational orientations of each residue in the second conformation 120. Denoising the noise frame 116 based on these updated S₂ features allows for the generation of an accurate second conformation 120. The generated first and second conformation 120s, when combined, form the motion trajectory of the protein from the time point corresponding to the first conformation 114 to the time point corresponding to the second conformation 120.

In this embodiment, by utilizing the attention module within the diffusion blocks 118, S₁ (encoding spatiotemporal information) and S₂ are jointly input to calculate attention weights. This enables the second residue features S₂ to specifically learn the temporal and spatial influences from residues in the first conformation 114 (e.g., the influence of the fifth residue in the first conformation 114 on the position and rotation of the second residue in the second conformation 120). This update method strictly adheres to the temporal sequence and spatial correlations of conformational evolution, helping to resolve the physical logic contradictions inherent in traditional methods, such as the separation of space and time and the non-causal “seeing” of future conformations by past ones, thereby enhancing the plausibility of conformational changes. Furthermore, by leveraging the backbone module of the diffusion blocks 118 to iteratively denoise the noise frame 116, combined with S₂ updated by the spatiotemporal information from S₁, an accurate second conformation 120 is generated. This can effectively suppress the accumulation of deviations in long-term prediction, thereby providing a reliable technical pathway for the continuous prediction of protein conformational dynamics trajectories.

It should be understood that an instance of the server 110 may be an independent physical server, a server cluster or distributed system composed of multiple physical servers, or a cloud server. Basic cloud computing services such as cloud services, cloud databases, cloud computing, cloud functions, cloud storage, network services, cloud communication, middleware services, domain name services, security services, CDN, as well as big data and artificial intelligence platforms are provided by such a cloud server. Connections between servers can be made directly or indirectly via wired or wireless communication means, which is not limited herein.

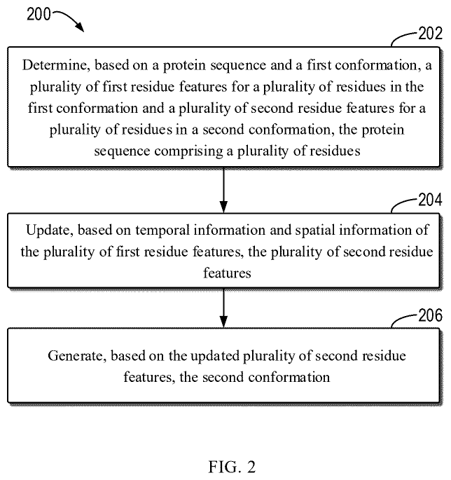

FIG. 2 shows a flowchart of a method 200 according to some embodiments of the present disclosure. In this embodiment, the method can be executed by the server 110 of the embodiment in FIG. 1. At block 202, based on a protein sequence and a first conformation, a plurality of first residue features for a plurality of residues in the first conformation and a plurality of second residue features for a plurality of residues in a second conformation are determined, where the protein sequence includes a plurality of residues. In some embodiments, the protein sequence is the sequence of amino acids forming the protein arranged in a specific order, serving as the foundation for protein structure and function, and for example contains information such as the name and chemical properties (e.g., polarity, hydrophobicity) of each residue. For example, a protein sequence might be “Met-Ala-Ser-Glu-Leu”, representing five residues in order: Methionine (Met), Alanine (Ala), Serine (Ser), Glutamic Acid (Glu), and Leucine (Leu). In a protein sequence, after each amino acid forms a peptide chain, it loses some atoms, and the remaining part is referred to as a residue. In some embodiments, the first conformation is the starting state for protein conformation prediction, i.e., the three-dimensional spatial structure of the protein at a specific time point, containing the three-dimensional coordinates (x, y, z) and rotational orientation (e.g., orientation in Rotation-space) of all residues. For example, in the first conformation of a protein, residue 3 (Ser) might have coordinates (2.1, 3.5, 4.2) and a rotational orientation at a specific 30-degree angle to the x-axis in Rotation-space. In some embodiments, the first residue features refer to the feature vectors obtained by encoding each residue in the first conformation, including spatial information of that residue. Similarly, in some embodiments, the second residue features refer to the initial feature vectors for each residue in the second conformation to be predicted, which are generated by jointly encoding the protein sequence and the first conformation, and serve as the basis for subsequent updates.

At block 204, the plurality of second residue features are updated based on the temporal information and spatial information of the plurality of first residue features. In some embodiments, through methods such as an attention mechanism, the second residue features (S₂) are enabled to learn the temporal sequence logic and spatial interactions contained within the first residue features (S₁), thereby correcting the feature representation of S₂. In this way, each residue in the second conformation can, within the attention module, attend to the position and rotational orientation of each residue in the first conformation at its starting time point, allowing the update of the plurality of second residue features to consider the influence of each residue in the first conformation. For example, the position and rotational orientation of the fifth residue in the first conformation may influence the position and rotational orientation of the second residue in the second conformation. In some embodiments, the temporal information may be incorporated in to S₁ by adjusting S₁ (e.g., rotating S₁ by one degree). In some embodiments, the temporal information may be represented by a feature related to S₁.

At block 206, the second conformation is generated based on the updated plurality of second residue features. In some embodiments, utilizing models such as a diffusion model, a noise frame is iteratively denoised, combined with the updated second residue features that contain precise spatiotemporal correlations, to output the conformation of the protein at a subsequent time point. In this step, the plurality of second residue features are operating objects.

The method involves updating the residues of the second conformation based on both the temporal information and spatial information of each residue in the first conformation. The second conformation generated in this manner is not only structurally reasonable itself, but the transition from the first conformation also more closely conforms to real physical dynamics laws. This directly enhances the accuracy of the second conformation. Moreover, since this method operates based on residue features, computational and memory overheads are reduced to some extent, enabling the prediction of conformations for larger proteins or over longer time scales.

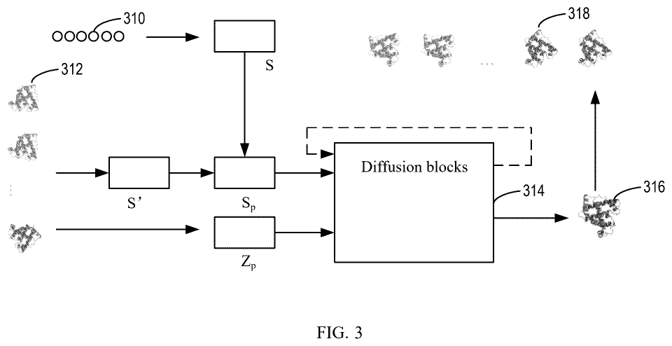

FIG. 3 shows a schematic diagram of a process for generating a conformation according to an embodiment of the present disclosure. In some embodiments, a plurality of base residue features are determined based on the protein sequence, where the plurality of base residue features indicate properties and an order of the plurality of residues. In this embodiment, an encoder can be used to encode the protein sequence 310 to extract a plurality of base residue features S therefrom. The base residue features S are an abstract representation of the residues, including at least information about the order of the residue in the protein sequence 310 and properties of the residue. Without considering the protein's structure, the plurality of base residue features S provide a highly precise representation of the residues.

To supplement information regarding the spatial structure of the residues and their information at different time points, historical conformations 312 are referenced. The historical conformations 312 include all previously generated conformations and a pre-provided first conformation. Each conformation in the historical conformations corresponds to a specific time point. In some embodiments, a preset time interval exists between consecutive conformations, and each conformation is marked with a sequence number, allowing the specific time point corresponding to a conformation to be inferred based on its sequence number and the preset time interval. In some embodiments, it is assumed that the historical conformations 312 include the pre-provided first conformation.

In some embodiments, the plurality of first residue features S₁ for the first conformation and a plurality of initial features S′ for the second conformation are determined based on the first conformation. The plurality of first residue features S₁ indicate spatial information of the plurality of residues in the first conformation. By adjusting S₁ in a various manner, the temporal information may be incorporated into S₁. Based on the first conformation, an encoder can also be used to predict the plurality of initial features S′ for the plurality of residues in the second conformation. In some embodiments, pairwise feature may be introduced into the decoding process, to facilitate improving the accuracy of the decoding process. Pairwise feature represents relationships among a plurality of residues in a conformation. In some embodiments, the plurality of second residue features S₂ for the second conformation and pairwise feature Z₂ representing relationships among the plurality of residues in the second conformation are determined based on the plurality of base residue features S and the plurality of initial residue features S′. The pairwise feature Z₂ include a plurality of features, where one feature indicates the influence of one residue on another residue in the second conformation. Assuming the protein includes 3 residues, the pairwise feature Z₂ include 3×3=9 features, representing the influences between residues 1-3 and residues 1-3. In some embodiments, the pairwise feature Z₂ indicate distances between carbon beta atoms of the plurality of residues in the second conformation; for example, one feature within Z₂ indicates the distance between the carbon beta atom of one residue and the carbon beta atom of another residue in the second conformation.

Similarly, after the conformation generation process is repeated multiple times, the historical conformations 312 can include p-1 conformations (as shown in the figure), where p is a positive integer greater than or equal to 2. During the process of generating the p-th conformation 316, an encoder can be used to encode the historical conformations 312 to generate the plurality of initial features S′ for the p-th conformation 316. The plurality of residue features Sₚ for the p-th conformation 316 and the pairwise feature Zₚ representing relationships among the plurality of residues in the p-th conformation 316 are determined based on the plurality of base residue features S and the plurality of initial features S′ for the p-th conformation 316.

The plurality of residue features Sₚ for the p-th conformation 316 and the pairwise feature Zₚ representing the p-th conformation 316 are input into a plurality of diffusion blocks 314 to denoise a noise frame. The p-th conformation 316 is generated through an iterative denoising process (as indicated by the dashed lines), and this p-th conformation 316 is added to the historical conformations 312 to obtain updated historical conformations 318. In some embodiments, these updated historical conformations 318 can be used as the historical conformations for predicting the next conformation. The updated historical conformations 318 can also be processed to generate an animation for displaying the motion trajectory of the protein.

In this embodiment, encoding the protein sequence 310 using an encoder to generate the base residue features S allows for precise capture of residue properties (e.g., polarity, hydrophobicity) and sequence order information, providing stable and accurate foundational information for subsequent conformation prediction. Furthermore, when generating the p-th conformation 316, encoding the initial features S′ based on the historical conformations enables the new conformation prediction to fully utilize the temporal and spatial evolution patterns from the historical conformations, strengthening the temporal correlation of the conformation sequence and providing support in the time dimension for the continuous prediction of long-term protein motion trajectories. Additionally, generating the plurality of residue features Sₚ based on the base residue features S and the initial features S′, while simultaneously constructing the pairwise feature Zₚ (e.g., distances between carbon beta atoms of residues) representing inter-residue relationships, makes the prediction of the spatial structure of the conformation more aligned with the rules of interaction between residues.

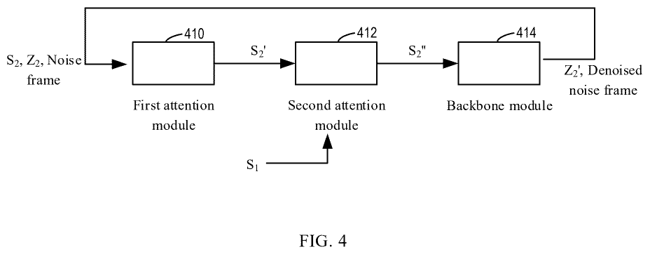

FIG. 4 shows a schematic diagram of a diffusion process according to an embodiment of the present disclosure. This diffusion process can involve a plurality of diffusion blocks having the same structure, which may be the plurality of diffusion blocks in FIG. 3. In some embodiments, the diffusion block includes a first attention module 410, a second attention module 412, and a backbone module 414. In this embodiment, the diffusion process for generating a second conformation based on a first conformation is described as an example, where a preset time interval exists between the first conformation and the second conformation.

In some embodiments, a noise frame for the second conformation can first be acquired. This noise frame can be a randomly generated noise frame used for generating the second conformation. In some embodiments, the plurality of second residue features can be adjusted based on the preset time interval, the noise frame, and the pairwise feature. For example, the noise frame, the pairwise feature Z₂, and the plurality of second residue features S₂ can be input into the first attention module 410. The noise frame and the pairwise feature Z₂ are used to adjust the plurality of second residue features S₂, enabling S₂ to attend to the intra-frame structure within the noise frame and to the interactions between residues indicated by the pairwise feature Z₂. In some embodiments, one or more modules of the model have a conditioning channel, and a preset time interval exists between the first conformation and the second conformation and can be input into this conditioning channel, which can improve the accuracy of the generated conformation. In some embodiments, the plurality of first residue features may be adjusted based on an order of the first conformation to obtain the adjusted plurality of first residue features S₁′. In some embodiments, the plurality of first residue features may be rotated by a preset degree to reflect the temporal information. For example, rotating the plurality of first residue features by p degrees may guide the attention module to know that the first conformation is the p-th conformation that appears at p time intervals.

In some embodiments, attention weights of each residue in the second conformation relative to each residue in the first conformation and to each residue in the second conformation are determined based on the adjusted plurality of first residue features S₁′ and the adjusted plurality of second residue features S₂'. For example, the adjusted plurality of first residue features S₁′ and the adjusted plurality of second residue features S₂′ can be input into the second attention module 412. Based on the Q vectors and K vectors within the second attention module 412, computations are performed on S₁′ and S₂′ to obtain the attention weights. In some embodiments, the attention scores of S₂′ relative to S₁′ and S₂′ itself are calculated by Q×K^{T} (e.g., the attention degree of residue 2 in S₂′ to residue 5 in S₁'). In some embodiments, the attention scores are normalized to obtain the attention weights.

In some embodiments, the plurality of second residue features S₂ are updated based on the attention weights and the adjusted plurality of second residue features S₂′ to obtain the updated plurality of second residue features S₂″. For example, a weighted sum is performed on the weights using the V vectors within the second attention module 412, resulting in the updated plurality of second residue features S₂″ that integrate the temporal and spatial information from S₁′.

In some embodiments, the second conformation is generated by denoising the noise frame based on the updated plurality of second residue features S₂″. For example, the updated plurality of second residue features S₂″, the pairwise feature Z₂, and the noise frame are input into the backbone module 414. The backbone module 414 denoises the noise frame based on the S₂″ and Z₂, and updates the pairwise feature Z₂ to obtain Z₂′.

Regarding the denoising process, in some embodiments, a denoising vector is determined based on the updated plurality of second residue features S₂″, where the denoising vector indicates a denoising direction and a denoising speed. The noise frame is a representation of the protein conformation in its current state. At the start of the diffusion process, it can be pure random noise. As denoising progresses, it gradually incorporates more real structural information. The denoising vector is the vector used to restore the noise frame to the protein conformation. Starting from the noise frame, this vector points towards the final protein conformation with a specific direction and magnitude. To improve the accuracy of the diffusion process, the denoising vector can be continuously updated through iterative operations.

In some embodiments, the following operations are iteratively performed until a preset stopping condition is met: a denoised noise frame is determined by denoising the noise frame according to the denoising vector; and the denoising vector is updated based on the time interval, the denoised noise frame, the updated pairwise feature, and the updated plurality of second residue features. In this iterative operation, the denoising process of the noise frame is divided into multiple stages, with the noise frame being partially denoised each time, making the restoration of the noise frame more accurate. Each time the denoising vector is updated, the plurality of second residue features are referenced, ensuring that the generated protein conformation is reasonable and accurate. In some embodiments, the denoised noise frame is determined as the second conformation. In some embodiments, after the second conformation is generated in the final iteration, the updated plurality of second residue features from this final iteration can serve as the features that best represent the intra-frame structure of the second conformation. Thus, when generating a third conformation (a conformation subsequent to the second conformation by the preset time interval), these updated second residue features from the final iteration (e.g., pre-cached) can be directly used as the features S₂.

In some cases, factors such as overfitting can reduce the accuracy of the generated protein conformation. To address this, small random noise can be added to intermediate products. In some embodiments, during the denoising process of determining a denoised noise frame by denoising the noise frame according to the denoising vector, an initial noise frame can be determined based on the denoising vector and the noise frame. That is, denoising is first performed to a certain extent according to the denoising vector. In some embodiments, the denoised noise frame is determined based on the initial noise frame and random noise. After denoising, a small amount of random noise is added to the initial noise frame, which can mitigate negative effects caused by factors like overfitting and exposure bias.

In some embodiments, a plurality of third residue features are determined based on the protein sequence and historical conformations, where the historical conformations include the first conformation and the second conformation (i.e., when generating the third conformation, the historical conformation has accumulated p-1=2 conformations). In some embodiments, the protein sequence is re-encoded by an encoder, or the previously generated base residue features S are directly reused (these features having already accurately captured residue properties such as polarity, hydrophobicity, and sequence order information, thus avoiding redundant encoding for improved efficiency). In some embodiments, the historical conformations comprising the first and second conformations are jointly encoded by an encoder to generate a plurality of initial features for the third conformation. These initial features for the third conformation integrate the temporal evolution patterns (e.g., trends in residue position changes, logic of rotational orientation adjustments) and spatial evolution information (e.g., dynamic changes in inter-residue interactions) from the first conformation to the second conformation. In some embodiments, based on the plurality of base residue features S and the initial features, the plurality of third residue features S₃ are determined through feature fusion (e.g., concatenation, weighted summation), while pairwise feature Z₃ representing relationships among the plurality of residues in the third conformation are constructed. Z₃ can indicate interaction information between any two residues in the third conformation, such as the distance between the carbon beta atom of residue i and the carbon beta atom of residue j.

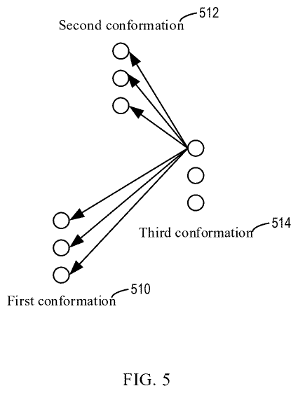

In some embodiments, the plurality of third residue features are updated by determining, based on a preset time interval, attention weights of each residue in the third conformation relative to each residue in the first conformation, to each residue in the second conformation, and to each residue in the third conformation. FIG. 5 shows a schematic diagram illustrating the effect of joint attention according to an embodiment of the present disclosure. In this embodiment, within the second attention module, each residue in the third conformation 514 can attend to the temporal information and spatial information of each residue in the first conformation 510, as well as the temporal information and spatial information of each residue in the second conformation 512. Thus, the third residue features can be updated to more accurate third residue features by calculating attention scores. In some embodiments, the third conformation is generated based on the updated plurality of third residue features. The updated third residue features, the pairwise feature Z₃, and a noise frame are input into the backbone module of a diffusion block, and the third conformation is generated by the backbone module.

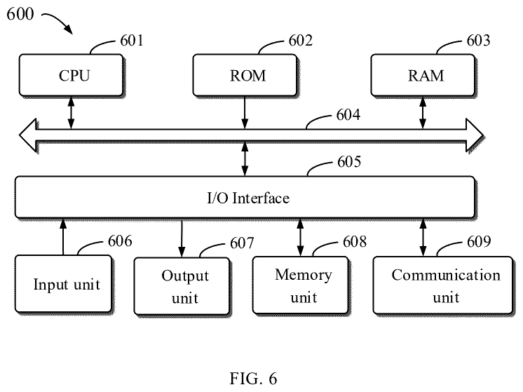

FIG. 6 illustrates a simplified block diagram of a device 600 that is suitable for implementing some example embodiments of the present disclosure. As illustrated therein, the device 600 includes a central processing unit (CPU) 601 that may perform various appropriate actions and processing based on computer program instructions stored in a Read-Only Memory (ROM) 602 or loaded from a memory unit 608 to a Random-Access Memory (RAM) 603. In the RAM 603, there may further store various programs and data needed for operations of the device 600. The CPU 601, ROM 602 and RAM 603 are connected to each other via a bus 604. An input/output (I/O) interface 605 is also connected to the bus 604.

Various components in the device 600 are connected to the I/O interface 605, including: an input unit 606 such as a keyboard, a mouse and the like; an output unit 607 such as various types of displays and loudspeakers, etc. ; a memory unit 608 such as a magnetic disk, an optical disk, and etc. ; and a communication unit 609 such as a network card, a modem, and a wireless communication transceiver, etc. The communication unit 609 allows the device 600 to exchange/formation/data with other devices via a computer network such as the Internet and/or various types of telecommunications networks. It is understood that the present disclosure may display, via the output unit 607, real-time dynamic change information of the customer satisfaction, key factor identification information of a group of customers or individual customers subjected to the satisfaction, optimized strategy information, and strategy implementation effect assessment information, etc.

The processing unit 601 may be implemented by one or more processing circuits. The processing unit 601 may be configured to perform various processes and processing described above. For example, in some embodiments, the process described above may be implemented as a computer software program that is tangibly embodied on a machine readable medium, e.g., the memory unit 608. In some embodiments, part or all of the computer program may be loaded and/or mounted onto the device 600 via ROM 602 and/or communication unit 609. When the computer program is loaded to the RAM 603 and executed by the CPU 601, one or more steps of the process as described above may be executed.

It is to be understood that although FIG. 6 is illustrated as an illustrative device to perform the process or method illustrated above, the embodiments of the present disclosure may also be implemented at one or more quantum computers, the present disclosure does not limit this aspect.

The present disclosure may be implemented a system, a method and/or a computer program product. The computer program product may comprise a computer-readable storage medium on which computer-readable program instructions for executing various aspects of the present disclosure are loaded.

The computer readable storage medium may be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium comprises the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform various aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processing unit of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the drawings illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It is also to be noted that, in some alternative implementations, the functions noted in the block may occur out of the order noted in the figures. For example, two blocks illustrated in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

In one aspect, there is provided a method, such as a computer-implemented method. The method comprises: determining, based on a protein sequence and a first conformation, a plurality of first residue features for a plurality of residues in the first conformation and a plurality of second residue features for a plurality of residues in a second conformation, the protein sequence comprising a plurality of residues; updating, based on temporal information and spatial information of the plurality of first residue features, the plurality of second residue features; and generating, based on the updated plurality of second residue features, the second conformation.

In some implementations, determining, based on the protein sequence and the first conformation, the plurality of first residue features for the plurality of residues in the first conformation and the plurality of second residue features for the plurality of residues in the second conformation comprises: determining, based on the protein sequence, a plurality of base residue features, wherein the plurality of base residue features indicate properties and an order of the plurality of residues; determining, based on the first conformation, the plurality of first residue features and a plurality of initial features for the second conformation; and determining, based on the plurality of base residue features and the plurality of initial features, the plurality of second residue features and pairwise feature representing relationships among the plurality of residues in the second conformation.

In some implementations, a preset time interval exists between the first conformation and the second conformation, and updating, based on temporal information and spatial information of the plurality of first residue features, the plurality of second residue features comprises: acquiring a noise frame for the second conformation; adjusting the plurality of second residue features based on the preset time interval, the noise frame, and the pairwise feature; adjusting the plurality of first residue features based on an order of the first conformation; determining attention weights of each residue in the second conformation relative to each residue in the first conformation and to each residue in the second conformation based on the adjusted plurality of first residue features and the adjusted plurality of second residue features; and updating the plurality of second residue features based on the attention weights and the adjusted plurality of second residue features.

In some implementations, generating the second conformation based on the updated plurality of second residue features comprises: generating the second conformation by denoising the noise frame based on the updated plurality of second residue features.

In some implementations, the method further comprises: updating the pairwise feature based on the updated plurality of second residue features.

In some implementations, generating the second conformation by denoising the noise frame based on the updated plurality of second residue features comprises: determining a denoising vector based on the updated plurality of second residue features, wherein the denoising vector indicates a denoising direction and a denoising speed; iteratively performing the following operations until a preset stopping condition is met: determining a denoised noise frame by denoising the noise frame according to the denoising vector; and updating the denoising vector based on the time interval, the denoised noise frame, the updated pairwise feature, and the updated plurality of second residue features; and determining the denoised noise frame as the second conformation.

In some implementations, the pairwise feature indicate distances between carbon beta atoms of the plurality of residues in the second conformation.

In some implementations, the method further comprises: determining, based on the protein sequence and a historical conformation, a plurality of third residue features, wherein the historical conformation comprises the first conformation and the second conformation; updating the plurality of third residue features by determining, based on a preset time interval, attention weights of each residue in a third conformation relative to each residue in the first conformation, to each residue in the second conformation, and to each residue in the third conformation; and generating the third conformation based on the updated plurality of third residue features.

In some implementations, the method is performed by a model having a conditioning channel, and a preset time interval exists between the first conformation and the second conformation and is input into the conditioning channel.

In another aspect, there is provided an electronic device. The electronic device comprises: at least one display; at least one memory; and at least one processor coupled with the at least one memory and configured to cause the device to: determine, based on a protein sequence and a first conformation, a plurality of first residue features for a plurality of residues in the first conformation and a plurality of second residue features for a plurality of residues in a second conformation, the protein sequence comprising a plurality of residues; update, based on temporal information and spatial information of the plurality of first residue features, the plurality of second residue features; and generate, based on the updated plurality of second residue features, the second conformation.

In some implementations, the electronic device is further caused to: determine, based on the protein sequence, a plurality of base residue features, wherein the plurality of base residue features indicate properties and an order of the plurality of residues; determine, based on the first conformation, the plurality of first residue features and a plurality of initial features for the second conformation; and determine, based on the plurality of base residue features and the plurality of initial features, the plurality of second residue features and pairwise feature representing relationships among the plurality of residues in the second conformation.

In some implementations, a preset time interval exists between the first conformation and the second conformation, and the electronic device is further caused to: acquire a noise frame for the second conformation; adjust the plurality of second residue features based on the preset time interval, the noise frame, and the pairwise feature; adjust the plurality of first residue features based on an order of the first conformation; determine attention weights of each residue in the second conformation relative to each residue in the first conformation and to each residue in the second conformation based on the adjusted plurality of first residue features and the adjusted plurality of second residue features; and update the plurality of second residue features based on the attention weights and the adjusted plurality of second residue features.

In some implementations, the electronic device is further caused to: generate the second conformation by denoising the noise frame based on the updated plurality of second residue features.

In some implementations, the electronic device is further caused to: update the pairwise feature based on the updated plurality of second residue features.

In some implementations, the electronic device is further caused to: determine a denoising vector based on the updated plurality of second residue features, wherein the denoising vector indicates a denoising direction and a denoising speed; iteratively performing the following operations until a preset stopping condition is met: determine a denoised noise frame by denoising the noise frame according to the denoising vector; and update the denoising vector based on the time interval, the denoised noise frame, the updated pairwise feature, and the updated plurality of second residue features; and determine the denoised noise frame as the second conformation.

In some implementations, the pairwise feature indicate distances between carbon beta atoms of the plurality of residues in the second conformation.

In some implementations, the electronic device is further caused to: determine, based on the protein sequence and a historical conformation, a plurality of third residue features, wherein the historical conformation comprises the first conformation and the second conformation; update the plurality of third residue features by determining, based on a preset time interval, attention weights of each residue in a third conformation relative to each residue in the first conformation, to each residue in the second conformation, and to each residue in the third conformation; and generate the third conformation based on the updated plurality of third residue features.

In some implementations, the method is performed by a model having a conditioning channel, and a preset time interval exists between the first conformation and the second conformation and is input into the conditioning channel.

In a further aspect, there is provided a non-transitory computer-readable storage medium comprising program instructions for causing an apparatus to: determine, based on a protein sequence and a first conformation, a plurality of first residue features for a plurality of residues in the first conformation and a plurality of second residue features for a plurality of residues in a second conformation, the protein sequence comprising a plurality of residues; update, based on temporal information and spatial information of the plurality of first residue features, the plurality of second residue features; and generate, based on the updated plurality of second residue features, the second conformation.

In some implementations, the apparatus is further caused to: determine, based on the protein sequence, a plurality of base residue features, wherein the plurality of base residue features indicate properties and an order of the plurality of residues; determine, based on the first conformation, the plurality of first residue features and a plurality of initial features for the second conformation; and determine, based on the plurality of base residue features and the plurality of initial features, the plurality of second residue features and pairwise feature representing relationships among the plurality of residues in the second conformation.

Although the present disclosure has been described in languages specific to structural features and/or methodological acts, it is to be understood that the present disclosure defined in the appended claims is not necessarily limited to the specific features or acts described above. Rather, the specific features and acts described above are disclosed as example forms of implementing the claims.

I/We claim:

1. A method, comprising:
determining, based on a protein sequence and a first conformation, a plurality of first residue features for a plurality of residues in the first conformation and a plurality of second residue features for a plurality of residues in a second conformation, the protein sequence comprising a plurality of residues;
updating, based on temporal information and spatial information of the plurality of first residue features, the plurality of second residue features; and
generating, based on the updated plurality of second residue features, the second conformation.

2. The method of claim 1, wherein determining, based on the protein sequence and the first conformation, the plurality of first residue features for the plurality of residues in the first conformation and the plurality of second residue features for the plurality of residues in the second conformation comprises:
determining, based on the protein sequence, a plurality of base residue features, wherein the plurality of base residue features indicate properties and an order of the plurality of residues;
determining, based on the first conformation, the plurality of first residue features and a plurality of initial features for the second conformation; and
determining, based on the plurality of base residue features and the plurality of initial features, the plurality of second residue features and pairwise feature representing relationships among the plurality of residues in the second conformation.

3. The method of claim 2, wherein a preset time interval exists between the first conformation and the second conformation, and updating, based on temporal information and spatial information of the plurality of first residue features, the plurality of second residue features comprises:
acquiring a noise frame for the second conformation;
adjusting the plurality of second residue features based on the preset time interval, the noise frame, and the pairwise feature;
adjusting the plurality of first residue features based on an order of the first conformation;
determining attention weights of each residue in the second conformation relative to each residue in the first conformation and to each residue in the second conformation based on the adjusted plurality of first residue features and the adjusted plurality of second residue features; and
updating the plurality of second residue features based on the attention weights and the adjusted plurality of second residue features.

4. The method of claim 3, wherein generating the second conformation based on the updated plurality of second residue features comprises:
generating the second conformation by denoising the noise frame based on the updated plurality of second residue features.

5. The method of claim 4, further comprising:
updating the pairwise feature based on the updated plurality of second residue features.

6. The method of claim 5, wherein generating the second conformation by denoising the noise frame based on the updated plurality of second residue features comprises:
determining a denoising vector based on the updated plurality of second residue features, wherein the denoising vector indicates a denoising direction and a denoising speed;
iteratively performing the following operations until a preset stopping condition is met:
determining a denoised noise frame by denoising the noise frame according to the denoising vector; and
updating the denoising vector based on the time interval, the denoised noise frame, the updated pairwise feature, and the updated plurality of second residue features; and
determining the denoised noise frame as the second conformation.

7. The method of claim 3, wherein the pairwise feature indicate distances between carbon beta atoms of the plurality of residues in the second conformation.

8. The method of claim 1, further comprising:
determining, based on the protein sequence and a historical conformation, a plurality of third residue features, wherein the historical conformation comprises the first conformation and the second conformation;
updating the plurality of third residue features by determining, based on a preset time interval, attention weights of each residue in a third conformation relative to each residue in the first conformation, to each residue in the second conformation, and to each residue in the third conformation; and
generating the third conformation based on the updated plurality of third residue features.

9. The method of claim 1, wherein the method is performed by a model having a conditioning channel, and a preset time interval exists between the first conformation and the second conformation and is input into the conditioning channel.

10. An electronic device comprising:
at least one processor; and
at least one memory storing instructions that, when executed by the at least one processor, cause the electronic device at least to:
determine, based on a protein sequence and a first conformation, a plurality of first residue features for a plurality of residues in the first conformation and a plurality of second residue features for a plurality of residues in a second conformation, the protein sequence comprising a plurality of residues;
update, based on temporal information and spatial information of the plurality of first residue features, the plurality of second residue features; and
generate, based on the updated plurality of second residue features, the second conformation.

11. The electronic device of claim 10, wherein the instructions to determine, based on the protein sequence and the first conformation, the plurality of first residue features for the plurality of residues in the first conformation and the plurality of second residue features for the plurality of residues in the second conformation, further cause the electronic device at least to:
determine, based on the protein sequence, a plurality of base residue features, wherein the plurality of base residue features indicate properties and an order of the plurality of residues;
determine, based on the first conformation, the plurality of first residue features and a plurality of initial features for the second conformation; and
determine, based on the plurality of base residue features and the plurality of initial features, the plurality of second residue features and pairwise feature representing relationships among the plurality of residues in the second conformation.

12. The electronic device of claim 11, wherein a preset time interval exists between the first conformation and the second conformation, and the instructions to update, based on temporal information and spatial information of the plurality of first residue features, the plurality of second residue features, further cause the electronic device at least to:
acquire a noise frame for the second conformation;
adjust the plurality of second residue features based on the preset time interval, the noise frame, and the pairwise feature;
adjust the plurality of first residue features based on an order of the first conformation;
determine attention weights of each residue in the second conformation relative to each residue in the first conformation and to each residue in the second conformation based on the adjusted plurality of first residue features and the adjusted plurality of second residue features; and
update the plurality of second residue features based on the attention weights and the adjusted plurality of second residue features.

13. The electronic device of claim 12, wherein the instructions to generate the second conformation based on the updated plurality of second residue features, further cause the electronic device at least to:
generate the second conformation by denoising the noise frame based on the updated plurality of second residue features.

14. The electronic device of claim 13, wherein the instructions further cause the electronic device at least to:
update the pairwise feature based on the updated plurality of second residue features.

15. The electronic device of claim 14, wherein the instructions to generate the second conformation by denoising the noise frame based on the updated plurality of second residue features, further cause the electronic device at least to:
determine a denoising vector based on the updated plurality of second residue features, wherein the denoising vector indicates a denoising direction and a denoising speed;
iteratively performing the following operations until a preset stopping condition is met:
determine a denoised noise frame by denoising the noise frame according to the denoising vector; and
update the denoising vector based on the time interval, the denoised noise frame, the updated pairwise feature, and the updated plurality of second residue features; and
determine the denoised noise frame as the second conformation.

16. The electronic device of claim 12, wherein the pairwise feature indicate distances between carbon beta atoms of the plurality of residues in the second conformation.

17. The electronic device of claim 10, wherein the instructions further cause the electronic device at least to:
determine, based on the protein sequence and a historical conformation, a plurality of third residue features, wherein the historical conformation comprises the first conformation and the second conformation;
update the plurality of third residue features by determining, based on a preset time interval, attention weights of each residue in a third conformation relative to each residue in the first conformation, to each residue in the second conformation, and to each residue in the third conformation; and
generate the third conformation based on the updated plurality of third residue features.

18. The electronic device of claim 10, wherein the method is performed by a model having a conditioning channel, and a preset time interval exists between the first conformation and the second conformation and is input into the conditioning channel.

19. A non-transitory computer-readable storage medium comprising program instructions for causing an apparatus to:
determine, based on a protein sequence and a first conformation, a plurality of first residue features for a plurality of residues in the first conformation and a plurality of second residue features for a plurality of residues in a second conformation, the protein sequence comprising a plurality of residues;
update, based on temporal information and spatial information of the plurality of first residue features, the plurality of second residue features; and
generate, based on the updated plurality of second residue features, the second conformation.

20. The non-transitory computer-readable storage medium of claim 19, wherein the program instructions to determine, based on the protein sequence and the first conformation, the plurality of first residue features for the plurality of residues in the first conformation and the plurality of second residue features for the plurality of residues in the second conformation, further cause the apparatus at least to:
determine, based on the protein sequence, a plurality of base residue features, wherein the plurality of base residue features indicate properties and an order of the plurality of residues;
determine, based on the first conformation, the plurality of first residue features and a plurality of initial features for the second conformation; and
determine, based on the plurality of base residue features and the plurality of initial features, the plurality of second residue features and pairwise feature representing relationships among the plurality of residues in the second conformation.
